(19) United States
(12) Plant Patent Application Publication
(10) Pub. No.: US 2003/0056262 P1
(43) Pub. Date: Mar. 20, 2003

(54) Chrysanthemum plant named 'Stellar Time'

(76) Inventor: Mark Roland Boeder, J H Den Haag (NL)

Correspondence Address: Mark K. Dickson, HOWREY SIMON ARNOLD & WHITE, LLP, 750 Bering Drive, Houston, TX, 77057-2198, US

(21) Appl. No.: 09/957,490

(22) Filed: Sep. 19, 2001

(51) Int. Cl. A01H 5/00

(52) U.S. Cl. PLT/288

(57) ABSTRACT

A new variety of Chrysanthemum plant named ‘Stellar Time,’ having an upright and branching growth habit with medium vigour and medium circular flat white flowers.

### LATIN NAME OF THE GENUS AND SPECIES

Botanical classification: Dendranthema grandiflora.

### VARIETY DENOMINATION

The new Chrysanthemum variety denomination is ‘Stellar Time.’

### BACKGROUND OF THE INVENTION

The present invention comprises a new and distinct cultivar of Chrysanthemum botanically known as Dendranthema grandiflora, and referred to by the cultivar name ‘Stellar Time.’

‘Stellar Time,’ identified as 20283-0 originated from a cross made by Mark Boeder, in a controlled breeding program in Chichester, West Sussex, United Kingdom. The female parent of the new variety is the variety ‘Jewel Time,’ and the male parent is the variety known as ‘Diamond,’ U.S. Plant Pat. No. 8059. The new variety ‘Stellar Time’ has been asexually reproduced by vegetative cuttings at Chichester, West Sussex, United Kingdom and the distinguishing characteristics are retained through successive generations of asexual reproduction.

### BRIEF SUMMARY OF THE INVENTION

‘Stellar Time’ is a pot type of Chrysanthemum plant variety having fresh looking white decorative flowers with a yellow centre forming a flat capitulum.

### Comparison with Parent

Plants of the new Chrysanthemum variety ‘Stellar Time’ are dissimilar to the parent cultivar ‘Jewel Time’ in plant habitat and growth rate, being slightly more upright and free branching, with different colour flowers. In side-by-side comparisons in Chichester, West Sussex, United Kingdom, under commercial practice, plants of the new Chrysanthemum variety ‘Stellar Time’ differed from plants of the parent cultivar ‘Jewel Time’ in the following characteristics.

1. The new variety ‘Stellar Time’ produces white, flat flowers whereas the parent cultivar ‘Jewel Time’ produces creamy yellow, slightly reflexing flowers.

2. Plants of the new variety ‘Stellar Time’ have smaller and shorter inflorescence than plants of the parent cultivar ‘Jewel Time.’

3. On average the new variety ‘Stellar Time’ has a longer response time of about 7 days compared to ‘Jewel Time.’

4. ‘Stellar Time’ has darker green foliage compared to ‘Jewel Time.’

### Comparison with Other Varieties

Plants of the new Chrysanthemum variety ‘Stellar Time’ are dissimilar to the cultivar ‘Surf’ in plant habitat and growth rate. However, in side-by-side comparisons in Chichester, West Sussex, United Kingdom, under commercial practice, plants of the new Chrysanthemum variety ‘Stellar Time’ differed from plants of the cultivar ‘Surf’ in the following characteristics.

1. The new variety ‘Stellar Time’ produces white, flat flowers with a green centre, whereas the cultivar ‘Surf’ produces white, slightly reflexing flowers.

2. Plants of the new variety ‘Stellar Time’ have smaller and shorter inflorescence than plants of the parent cultivar ‘Surf.’

3. ‘Stellar Time’ has a longer response time of about three days compared to the variety ‘Surf.’

### BRIEF DESCRIPTION OF ILLUSTRATIONS

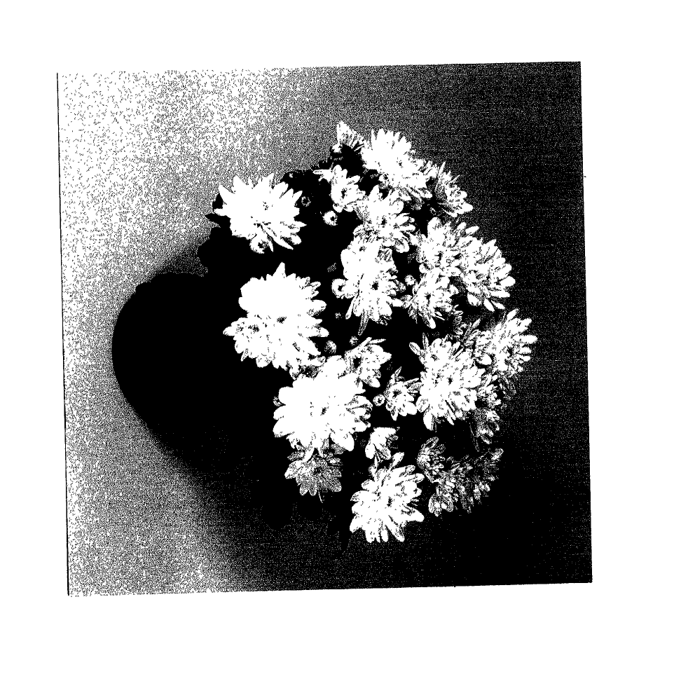

Typical specimens of the plant and flowers for the new Chrysanthemum variety ‘Stellar Time’ are shown in the accompanying photograph. The colours shown are as true as possible within the usual limits of this kind of illustration. FIG. 1 is a whole plant view of the new variety ‘Stellar Time’ grown in a pot. The plant shown in the illustration is 58 days from start of Short Days.

### DETAILED BOTANICAL DESCRIPTION

The following description of the new Chrysanthemum variety ‘Stellar Time’ is of plants grown in a greenhouse in Chichester, West Sussex, United Kingdom in the month of August. The cultivar has not been observed under all possible environmental conditions. The phenotype may vary significantly with variations in the environment such as temperature, length of day and light intensity, without any variance in genotype. The commercial classification of the new variety is a pot Chrysanthemum.

Plants of the new variety have been grown successfully under temperature conditions averaging about 18° C. at night and about 24° C. to 25° C. during the day under light conditions of about 5,000 to 6,000 foot candles. The plants respond well to the use of growth retardant, such as two B9 treatments at about 2000 ppm. To produce a commercial product the plants may be pinched once with the centre bud removed. The typical container size for commercial growth is 14 cm. It has been observed that the shelf life of the new variety is about 24 days with a response time of about 8 weeks. The new variety is suitable for growth in a temperature range of 16° C. to 25° C.

The new variety may be produced as a Spray and disbud. The following description is with respect to a plant produced as Spray pot Chrysanthemums. In the description of this new Chrysanthemum variety, colour values have been taken from The Royal Horticultural Society Colour Chart (R.H.S.C.C.).

### Plant

Plant type: Pot

Habit: Upright and branching

Height: Medium 18 to 20 cm

Width: Near 26 cm

Branching characteristics: Upright and spreading

Length of Lateral Branches: 8 to 12 cm

Number of Breaks from Pinch: 4 to 5

Stem Colour: 147 B

Response Time: 52 days

Vigour: Medium

Shelf Life: Near 24 days

Disease: No observations

Pest: No Observations

Growth retardant type and treatment: B9 85% 2 applications of 2 gram/litre at 2 days, 21 days after propagating unrooted vegetative cuttings.

The plants were grown for two weeks in Long Day conditions (20 hours of light) and then transferred to Short Day conditions (13 hours of dark).

Propagation:

Type.—Vegetative propagation via stem cuttings

Time to rooting.—12-14 days with soil temperatures of 18° C.

Rooting habit.—Fine and fibrous

### Foliage

Number of Leaves per Lateral Branch: 7 to 10

Compound or single: Single

Arrangement of Leaves: Alternate

Shape of Leaf.—Typically 5 lobed, oblong ovate

Size of Leaf.—Width (cm): Near 5 cm Length (cm): Near 10 cm

Leaf Apex.—Acute/Rounded

Base.—Obtuse

Attachment.—Petioled

Aspect.—Slightly undulating

Margin.—Palmately lobed

Surface characteristics.—Top: Pubescent Bottom: Pubescent

Petiole: Colour: Near 137 B Length: Near 2 cm

Venation: Net, prominent midvein under side.

Colour.—upper side: Near 147 D under side: Near 147 C

Colour: Mature Leaf, upper side: Near 137 A; under side: Near 137 B Young Leaf, upper side: Near 137 A; under side: Near 137 C

### Flower

Flower Appearance: Flat, circular

Flower type: Decorative

Flower Form: Flat

Flower shape: Circular

Flower Habit: Upright

Number of blossoms per branch: 3 to 5

Inflorescence form: Cyme

Depth of fully expanded blossoms.—Near 2 to 2½cm.

Diameter of fully expanded blossoms.—Near 5 to 8 cm.

Sepals:

Number.—19 to 23.

Colour.—Near 137 A.

Length.—Near 1.3 cm.

Width.—Near 0.2 cm.

Texture/Appearance.—Pubescent.

Peduncles: Peduncle length is shortest at the terminal flower and progressively longer moving down the stem; longest length is on average 10 cm; shortest length on average 6 cm.

Length.—8 cm.

Colour.—Near 137 B.

Surface.—Pubescent.

Habit.—Slightly undulating.

Strength.—Medium.

Pedicel:

Length. Terminal.—Near 4 cm; Lateral: Near 6 cm.

Colour.—Near 137 B.

Surface.—Pubescent.

Habit.—Slightly undulating.

Strength.—Medium.

Ray Florets:

Form/Shape.—Straight, elliptic.

Texture/Appearance.—Matte.

Number perflower.—200 to 220.

Length.—1½to 2.8 cm; Width: 0.4 to 0.8 cm.

Apex.—Round.

Base.—Tapered.

Margin.—Entire.

Disc florets:

Form/Shape.—Cylindrical.

Texture/Appearance.—Shiny.

Number per flower.—8 to 10.

Length.—Near 0.2 to 0.3 cm; Width: 0.05 to 0.1 cm.

Diameter of disc.—Near 0.3 cm.

Fragrance: Slight

Flower Bud (at onset of colour):

Length.—Near 0.7 cm.

Diameter.—Near 1.1 cm.

Form/shape.—Spherical.

General flower colour: White

1. Ray florets, upper side: Immature: Near 4 C Mature: Near 155 D Older/Fading: Near 155 B.

2. Ray florets, under side: Immature: Near 4 C Mature: Near 155 D Older/Fading: Near 155 B.

3. Disc florets: Immature: Near 8 C Mature: Near 138 D Older/Fading: Near 8 C.

4. Bud: Near 138 A

Flower progression: Flowers slightly cup and reflex, also slightly fade, during ageing.

### Reproductive Organs

Ray Florets: Pistillate

Pistil number.—100+.

Stigma colour.—Near 6 C.

Stigma shape.—Forked.

Style Colour.—Near 6 C.

Style Length.—Near 0.5 cm.

Disc Florets: Pistillate

Pistil number.—Near 2.

Stigma colour.—Near 6 C.

Stigma shape.—Forked.

Style Colour.—Near 6 C.

Style Length.—Near 0.1 to 0.2 cm.

Staminate.—no observation.

What is claimed is:

1. A new and distinct variety of Chrysanthemum plant, substantially as described and illustrated herein.
